(19) United States
(12) Patent Application Publication
(10) Pub. No.: US 2026/0123863 A1
(43) Pub. Date: May 7, 2026

(54) A Housing for Sample Collection

(71) Applicant: CAMBRIDGE ENTERPRISE LIMITED, Cambridge, Cambridgeshire (GB)

(72) Inventor: Alexander Patto, Cambridge (GB)

(21) Appl. No.: 19/117,163

(22) PCT Filed: Sep. 29, 2023
(86) PCT No.: PCT/EP2023/077122

§ 371 Date: Mar. 31, 2025

(30) Foreign Application Priority Data
Sep. 30, 2022 (GB) 2214398.6

(51) Int. Cl. A61B 5/15; A61B 5/157

(52) U.S. Cl. CPC A61B 5/150053; A61B 5/150022; A61B 5/150206; A61B 5/150358; A61B 5/150755; A61B 5/157

(57) ABSTRACT

A housing for sample collection is disclosed. The housing comprises an actuating member configured to move between a first position and a second position, a sample collector and an assay component. In the first position the sample collector is in contact with the assay component and in the second position the sample collector is separated from the assay component and configured to collect a sample.

### FIELD OF THE INVENTION

The present invention relates to a housing for sample collection and to a method of determining a concentration range of vitamin K. Specifically, the present invention relates to a housing for the collection of a bodily fluid for analysis using an assay.

### BACKGROUND

When performing an assay or test on a sample (for example, bodily fluid), it can be crucial that the assay steps are performed in the correct order, for example, if a buffer is required, the sample must be deposited before the buffer runs on the assay component, such as a lateral flow test strip. If the sample is added to the assay component without the buffer, or the buffer floods the assay component before the sample is added, the results of the assay or test may not be reliable. Unreliable results may influence diagnosis or treatment recommendations, and may require the test to be carried out again which can be distressing to a subject having the test, and also waste valuable resources. Further, the volume of sample for performing an assay or test can greatly influence the results. Therefore, there is a need to ensure a relatively consistent sample volume to improve results from semi-quantitative, or qualitative tests. Typically, to ensure consistent volumes of samples are collected, additional sterile apparatus are required.

Ensuring that a test is performed correctly can involve performing multiple complex steps manually, and a mistake or error in any one of these steps can result in unreliable test results. Currently, running an immunoassay, or lab based method as a point-of-care is highly complex and can involve multiple steps for sample collection, sample measurement, and buffer administration, before the sample and buffer mixture can be tested using an assay. This can introduce high costs, additional equipment and also errors. For a test to be most useful, it needs to be simple, quick, and accurate to reduce errors and lower operational costs. Thus, there is a need for a test that is highly automated in terms of sample collection and volume measurement, and buffer administration.

### SUMMARY

According to a first aspect of the invention, there is provided a housing for sample collection. The housing comprises an actuating member configured to move between a first position and a second position, a sample collector, and an assay component. In the first position the sample collector is in contact with the assay component and in the second position the sample collector is separated from the assay component and configured to collect a sample.

When the sample collector is in contact with the assay component, the contact allows for liquid communication between the sample collector and the assay component. For example, when the sample collector is in contact with the assay component, a sample (e.g. a bodily fluid, a bodily-derived fluid, or excretion from a human or other animal) may be passed from the sample collector to the assay component, for example, by capillary action.

When the sample collector is separated from the assay component, liquid communication between the sample collector and the assay component is not possible. The sample collector may be a pad, an ampule, a cup or a tube suitable for collecting the sample to be analysed.

The assay component may be, for example, a pad, an ampule, a cup or a tube suitable for holding reagents and/or analytes to perform an assay or test. The assay component may comprise or be adjacent to an assay test component. The assay may be, for example, a lateral flow assay or an enzyme linked immunosorbent assay (ELISA).

The sample collector may be separated from the assay component by a space or a barrier that prevents liquid communication between the sample collector and the assay component.

The housing may further comprise a needle. In the first position the needle may be disposed within the housing and in the second position the needle may protrude from an aperture in the housing.

In the first and/or second positions, the needle may or may not protrude through the sample collector. In the first position, the needle may be separated from the sample collector.

The sample collector may be a pad. The sample collector may be a fibre pad, for example, a wicking pad. The sample collector may be configured to hold a predetermined volume of liquid. The sample collector may collect the sample via capillary action.

The housing may further comprise an excess sample collector. The sample collector may be configured to hold a predetermined volume of liquid. When the actuator is in the second position, the excess sample collector may be configured to collect or absorb excess liquid from the sample collector once the sample collector holds its predetermined volume. The sample collector and the excess sample collector may be separated from the assay component when the actuator is in the second position. When the actuator is in the first position, the sample collector is separated from the excess sample collector.

The excess sample collector may be a pad, for example a tube, ampule, cup, or fibre pad, for example, a wicking pad.

The housing may comprise a window positioned to allow visual inspection of the sample collector and/or the excess sample collector.

The housing may further comprise a buffer component configured to receive a buffer, for example, from a reservoir. The buffer component may also be configured to be in contact with the sample collector in the first position and separated from the sample collection in the second position. The buffer component may be a pad, an ampule, a cup or a tube suitable for receiving and/or holding a buffer, e.g. a buffer solution, or further assay reagents. The buffer may be released from a buffer reservoir to allow it to move onto the buffer component. The release of the buffer from the buffer reservoir may be caused by the movement of the actuator from the first to the second position, for example, by a piercing member.

According to a second aspect of the invention, there is provided a housing for sample collection. The housing comprises an actuating member configured to move between a first position and a second position, a piercing member, and a needle. In the first position the needle is disposed within the housing and in the second position the needle protrudes from an aperture in the housing, and wherein the piercing member is configured to pierce a seal of a fluid reservoir upon movement from the first position to the second position.

Thus in one motion, for example, the actuating member being pressed by a digit, a needle can extend beyond the surface of the housing and is able to engage with the skin of a patient and a fluid, for example a buffer fluid or a test fluid, may be released from the fluid reservoir. The needle engaging with a patent's skin may pierce the skin releasing blood. The patient may be a neonate or infant.

There may be more than one needle. The needle, or plurality of needles may be attached to a needle bed. The needle may be a microneedle. The needle may be between 25 and 2000 μm in length. The needle may have a base diameter of between 25 and 500 μm. The needle may be a graded needle size, for example, 34 G. The needle may be solid or hollow.

The needle may protrude from the housing and the seal may be broken simultaneously. On release of pressure applied to the actuating member to move it from the first position to the second position, the actuating member may return to the first position. The return motion of the actuating member to the first position may be caused by, or aided by a spring.

The housing base or the housing cover may comprise the piercing member.

The assay component may be a lateral flow component, for example, a lateral flow test strip and/or a assay component.

The housing may comprise an assay component.

The needle may be disposed on a surface of the actuating member.

7. The housing may further comprise a sample collector configured to receive bodily fluid.

The sample collector may be, for example, a wicking pad, an absorbent pad, an absorbent gel, or other suitable means for collecting bodily fluid. The sample collector may surround the base of the needle. The sample collector may be configured to hold a predetermined volume of liquid, for example, via capillary action.

The bodily fluid may be blood, saliva or urine. The amount of bodily fluid collected (hereinafter also referred to as “a sample”) by the sample collector may be between 0.5 μm and 200 μm or between 0.5 μm and 100 μm. The sample may be between 1 μm and 10 μm, or between 5 μm and 10 μm.

Moving the actuating member from the first position to the second position may cause the needle to pierce the sample collector, or causes the needle to move past the sample collector.

The sample collector may be pierced at the same time or before the needle protrudes from the housing. This ensures that the body fluid sample (e.g. blood) collected by the housing once the needle has pierced the skin of a patient is collected by the sample collector.

The needle may be embedded into the actuating member, and be arranged so that the sample collector surrounds the needle, alternatively, the needle may be placed on top of the housing with a push fit, so that it lies on top of the sample collector.

The housing may comprise a base and a cover, and wherein the actuating member may be attached to, and moveable relative to, the base.

The base and cover are preferably separate sections or parts attached together by attaching means such as a plug and socket. The base and cover may be one integrated (e.g. moulded) structure which are attached to each other by a hinge. The actuating element is connected to the base, for example, by a hinge.

The housing may further comprise an adhesive arranged on a surface of the housing.

The adhesive may be arranged near to or around the aperture of the housing. The adhesive may be an annulus of adhesive around the aperture of the housing. The adhesive may be an adhesive film or tape. The adhesive may allow the location and securing of the housing onto a patient's skin.

The housing may further comprise an excess sample collector configured to receive bodily fluid from the sample collector or from a body.

Thus, the excess sample collector may receive excess bodily fluid from the sample collector, or directly from the body, thus ensuring that the sample collector is not flooded with an excess of bodily fluid which would affect or influence an analysis performed on or using the fluid, for example a lateral flow assay.

The housing may further comprise a sample collector aperture or a sample collector window in a surface of the housing configured to allow visual inspection of the sample collector.

The housing may further comprise a buffer fluid reservoir comprising a seal.

The fluid reservoir may comprise a buffer fluid or a test fluid. The fluid reservoir may be configured to be pierced by the piercing member when the actuating member is in the second position.

The housing may further comprise a buffer component configured to receive fluid from a, or the, buffer fluid reservoir.

The buffer component may be a cup, tube, ampule, or pad. The pad may be a fluid-absorbing pad, for example a fibrous pad, a gel pad and the like.

Once the actuating member has reached the second position, an end-stop or a spring may cause the needle to move to a needle position between the needle position in the first position and the needle position in the second position.

That is, the needle retracts from its most protruded position towards being within the housing, however the needle may still slightly protrude the housing.

The housing may further comprise an end-stop or spring to prevent the actuating member from moving past a predetermined position. For example, the needle may be retracted using a physical end-stop or a spring in which upon moving actuator from the first position to the second position causes the needle to retract.

The housing may further comprise a second spring configured to return the actuating member to the first position, or configured to move the actuating member, or a second actuating member, to a third position.

Retracting a needle slightly away from the patent skin may allow for better blood flow from the patient and easier sample collection. Certain types of needles may be better-suited to being retracted than others, for example, a hollow needle may not need retraction, whereas a solid needle may be more effective when retracted.

The actuating member may be configured to move to a third position, wherein moving the actuating member to the third position causes the needle to move to a needle position between the needle position in the first position and the needle position in the second position.

The third position may be beyond the second position in a linear direction between the first and second position. The third position may be linearly between the first and second position. The third position may be a position outside a straight line between the first and second position.

If the housing comprises a sample collector configured to receive a bodily fluid and at least one of an assay component or a buffer component configured to receive fluid from the buffer fluid reservoir, in the first position, the reservoir may be in contact with the assay component or the pad and in the second position, there may be a space between the reservoir and the assay component or the buffer component.

Thus, when the sample collector is receiving bodily fluid, it is physically separated from the buffer component or the assay component, and thus does not transfer the bodily fluid to the assay component.

When the actuating member returns to the first position, the sample collector, having received the bodily fluid, contacts either the buffer component, the lateral flow component, or both, allowing fluid communication between the sample collector and the buffer component and/or the assay component. Such an arrangement may allow fluid from the buffer fluid reservoir to flow via capillary action to the buffer component (e.g. a sample pad) and then both the fluid from the fluid reservoir and the bodily fluid to flow to the assay component (e.g. a lateral flow assay pad or lateral flow assay strip, alternatively, and ELISA assay component).

The housing may further comprise a lateral flow test strip viewing aperture or window configured to allow the viewing of a lateral flow test strip.

The lateral flow viewing aperture, for example, in a surface of the housing, allows the visual inspection of one or more test or control lines of a lateral flow component, for example a lateral flow test strip.

The housing may allow a bodily fluid sample of defined volume to be collected, or a visual check to determine whether insufficient sample has been collected, as well as buffer administration to be automatically delivered in sequence, by one press of a button on the lateral flow housing.

According to a third aspect of the invention, there is provided a test device comprising an assay component disposed in a housing according to the second aspect.

The assay component may comprise a lateral flow assay component. The assay component may comprise a lateral flow strip. The assay component may comprise a fluid (e.g. a test fluid or buffer fluid) reservoir.

The housing may comprise a blood separation filter, for example, a blood separation membrane FR-1 (0.35) from www.mdimembrane.com, disposed between the sample source and the assay component, for example between the sample collector and the assay component.

The assay component may be configured to detect the presence or absence of a vitamin. The detection of the presence or absence of a vitamin may be performed directly by testing for the present or absence of the vitamin itself, or it may be performed indirectly, by testing for the presence or absence of a marker associated with the vitamin.

The assay may include lateral-flow components containing anti-PIVKA-II antibodies for detection of PIVKA-II, a vitamin D lateral flow assay, a vitamin B12 lateral flow assay or a vitamin A lateral flow assay.

The lateral flow component may comprise a quantitative lateral flow component.

That is, the lateral flow test is able to give a quantitative reading of a component of an analyte, for example, the amount of vitamin K in a patient's blood.

According to a fourth aspect of the invention, there is provided a method of determining a concentration range of vitamin K in a sample using a PIVKA-II lateral flow assay. The method comprises performing a PIVKA-II lateral flow test on the sample, measuring the intensity of the test line on the lateral flow test strip, and determining a concentration range of vitamin K of the sample based on a test result indicator.

The test result indicator may be a line, the line may be more than one line (e.g. 2, 3, 4, 5, lines), the test result line may vary in intensity, the intensity may indicate the concentration of the analyte. The test result indicator may be a dot, or an array of dots.

The test result indicator may be a measure of, for example, conductivity. This may be performed as an alternative or in addition to a visual inspection of a test indictor. Can also be UV e.g. outside human visual range, could also be excited and then analysed (e.g. green fluorescent protein (GFP)).

The PIVKA-II lateral flow assay may be performed using a PIVKA-II assay component and test strip.

The sample used to perform the PIVKA-II lateral flow test may have a volume of between 0.5 μl and 60 μl.

The intensity of the test result indicator may be measured using absorbance.

The intensity of the test result indicator may be measured using an image, for example an image from a smartphone.

The method may further comprise comparing the intensity of the test line of the lateral flow strip against a predetermined threshold, or against a control test result indicator.

The predetermined threshold may be clinically-determined, for example, for PIVKA-II, may be below 51 mAU/mL.

According to a fifth aspect of the invention, there is provided a method of the fourth aspect performed using the housing of either the first, second or third aspect.

### BRIEF DESCRIPTION OF THE DRAWINGS

Certain embodiments of the present invention will now be described, by way of example, with reference to the accompanying drawings, in which:

FIG. 1 is a perspective view of a housing for sample collection;

FIG. 2 is a second perspective view of the housing for sample collection of FIG. 1;

FIG. 3 is a perspective view of a first part of a second housing for sample collection;

FIG. 4 is a perspective view of a second part of a second housing for sample collection;

FIG. 5 is a photograph of a third housing for sample collection;

FIG. 6 is a photograph of the operation of a third housing for sample collection;

FIG. 7 is a photograph of inside the third housing for sample collection;

FIGS. 8a to 8c are photographs of a sequence of buffer release using the third housing;

FIG. 9 is a perspective view of a solid microneedle;

FIG. 10 is a perspective view of an array of solid microneedles;

FIG. 11 is a perspective view of a hollow microneedle;

FIGS. 12a to 12d are schematic diagrams showing a collection of a sample using a fourth housing;

FIGS. 13a to 13d are schematic diagrams showing a collection of a sample using a fifth housing;

FIGS. 14a to 14d are schematic diagrams showing a collection of a sample using a sixth housing;

FIGS. 15a to 15d are schematic diagrams showing a collection of a sample using a seventh housing;

FIGS. 16a to 16d are schematic diagrams showing a collection of a sample using a seventh housing;

FIG. 17 is a bar chart of intensity scored of a lateral flow test strip;

FIG. 18 is a photograph of seven lateral flow test strips;

FIG. 19 is a graph showing the intensity of a lateral flow test strip for different concentrations of prothrombin induced by vitamin K absence-II (PIVKA-II);

FIG. 20 is a second graph showing the intensity of a lateral flow test strip for different concentrations of prothrombin induced by vitamin K absence-II (PIVKA-II);

FIG. 21 is a bar chart of intensity scored of a lateral flow test strip;

FIGS. 22a to 22b are photographs of lateral flow test strips;

FIGS. 23a and 23b are photographs of lateral flow test strips from lateral flow assays performed with different sample volumes;

FIGS. 24a to 24g are photographs of lateral flow test strips from lateral flow assays performed with different samples with the same volumes;

FIG. 25 is a bar chart of intensity in arbitrary units of test lines normalised to a control line at different concentrations of prothrombin induced by vitamin K absence-II;

FIG. 26 is a graph of intensity in arbitrary units against concentration of prothrombin induced by vitamin K absence-II.

FIG. 27 is a graph of intensity in arbitrary units against concentration of prothrombin induced by vitamin K absence-II.

### DETAILED DESCRIPTION

### Housing

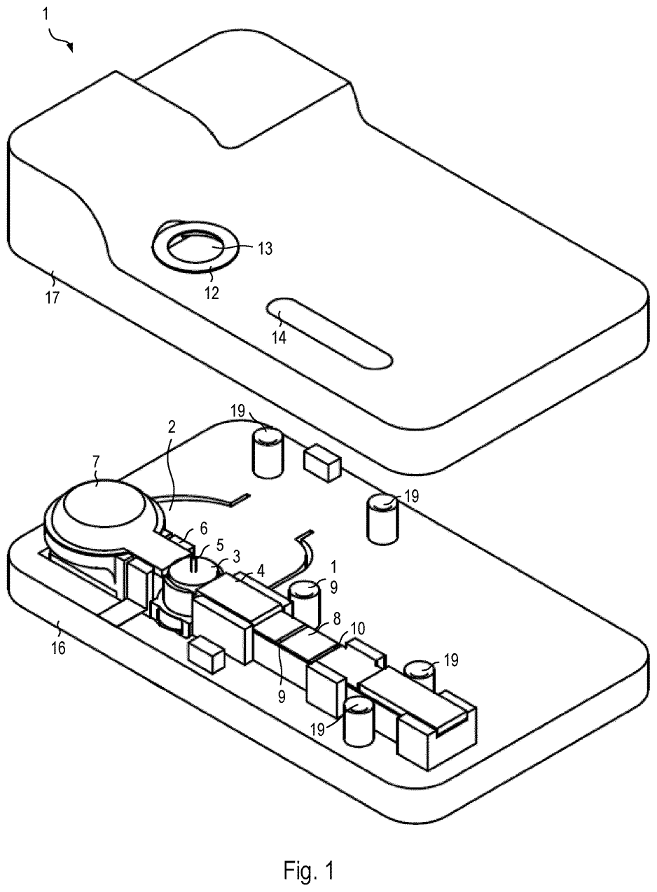

Referring to FIG. 1, a housing 1 for sample collection is shown. The housing 1 comprises an actuating member 2 which can be moved between a first position and a second position. The housing further comprises a sample collector 3 and an assay component 4. The assay component 4 may be, for example, a lateral flow assay pad or strip, or an enzyme linked immunosorbent assay (ELISA).

The housing 1 may comprise a needle 5, the needle 5 may be a solid or hollow needle. The needle 5 may be a microneedle. The needle 5 may be between 25 and 2000 μm in length. The needle may have a base diameter of between 25 and 500 μm. The needle may be a graded needle size, for example, 34 G. The needle 5 may be connected to the actuating member 2. The needle 5 may be arranged such that the sample collector 3 surrounds the needle 5. The housing 1 may include a buffer component 6 and a buffer reservoir 7 suitable for containing a buffer solution. The buffer component 6 is suitable for receiving the buffer solution from the buffer reservoir. The buffer solution may be a buffer solution which can be mixed with a sample from the sample collector before an assay is performed on the assay component 4. The buffer reservoir 7 may be a tube, ampule, or other container suitable for containing a buffer solution. The buffer reservoir 7 may have a seal (not shown) such as a foil seal, a plastic seal and the like, which may be broken to release the buffer solution onto or into the buffer component 6.

The housing 1 may also include a test strip 8 connected to the assay component 4 for performing a test, such as a lateral flow test. The test strip 8 may include a control indicator 9 to indicate whether the test has been performed correctly, and a test indicator 10 to indicate the result of a test.

The housing 1 may include an excess sample collector 12 which may be configured to collect excess sample above a predetermined volume from the sample collector 3. The housing may include a sample collector window or aperture 13 for visually inspecting or viewing the sample collector 3. A user may then look at the sample collector 3 to ensure it has the required volume or amount of sample in or on it. The housing may also include a test inspection window or aperture 14 through which the control indicator 9 and/or the test indicator may be viewed.

The housing 1 may be formed from a first part 16 and a second part 17. The first part 16 may form a base layer on which the sample collector 3, the assay component 4, the needle 5, the buffer component 6, the buffer reservoir 7, and the test strip 8 are located or attached. The actuating member 2 may be integrated into the first part 16. The second part 17 may form a top layer or case and may include the excess sample collector 12, the sample collector window or aperture 14 and the test window or aperture 14. The first and second parts 16, 17 may be connected together using any suitable means, for example, using plugs (not shown) and sockets 19 arranged to connect the first and second parts 16, 17 when pushed together. An adhesive (not shown) may be disposed on the surface of the second part 17. The adhesive may be arranged to allow for it to adhere to a sample source (not shown), for example, skin, to steady the housing to improve sample collection.

The sample collector 3, the assay component 4, the buffer component 6 and the excess sample collector 12 may be any suitable component for holding a sample, assay reagents and analytes, buffer, for example, a vessel, a pad, for example a fibrous pad, or a gel.

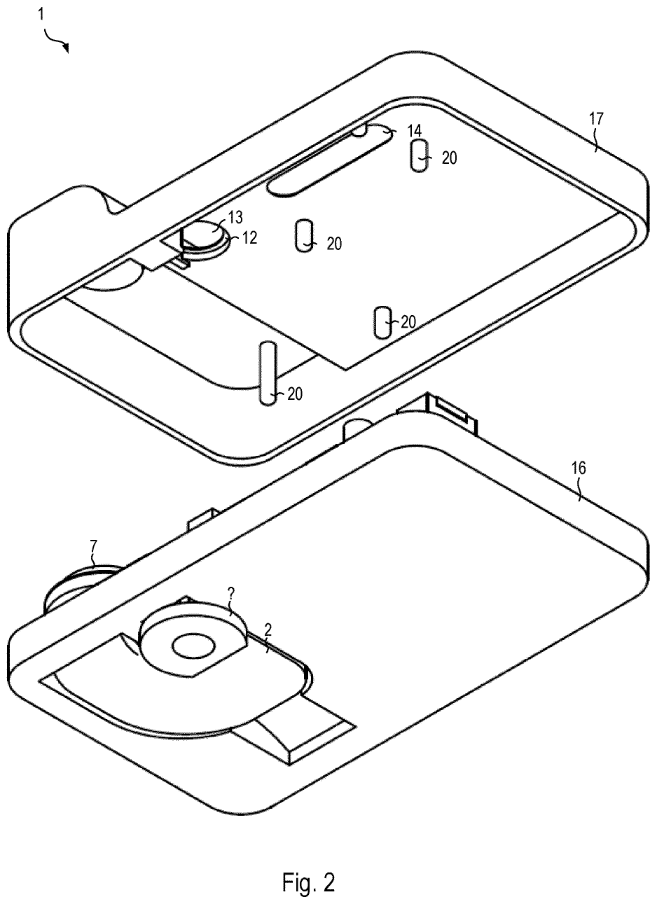

Referring also to FIG. 2, the sample collector housing 1 of FIG. 1 is shown from a second perspective. Plugs 20 are visible on the second part 17 which engage with the sockets 19 referenced in FIG. 1.

In a standard sample collection housing, for example, a lateral flow test device, the sample collector, and assay component are in a fixed position relative to each other. Likewise, a buffer component, if present is also in a fixed position relative to the sample collector. In the present housing 1, when the actuating member 2 is in the first position, the sample collector 3 is in contact (e.g. fluid, or liquid, communication) with the assay component 4 and/or the buffer component 6, and when the actuating member 2 is in the second position, the sample collector is separated from the assay component 4 and/or the buffer component 6, for example, either by a space or by a barrier that prevents fluid or liquid communication between the sample collector 3 and the assay component 4 and the buffer component 6.

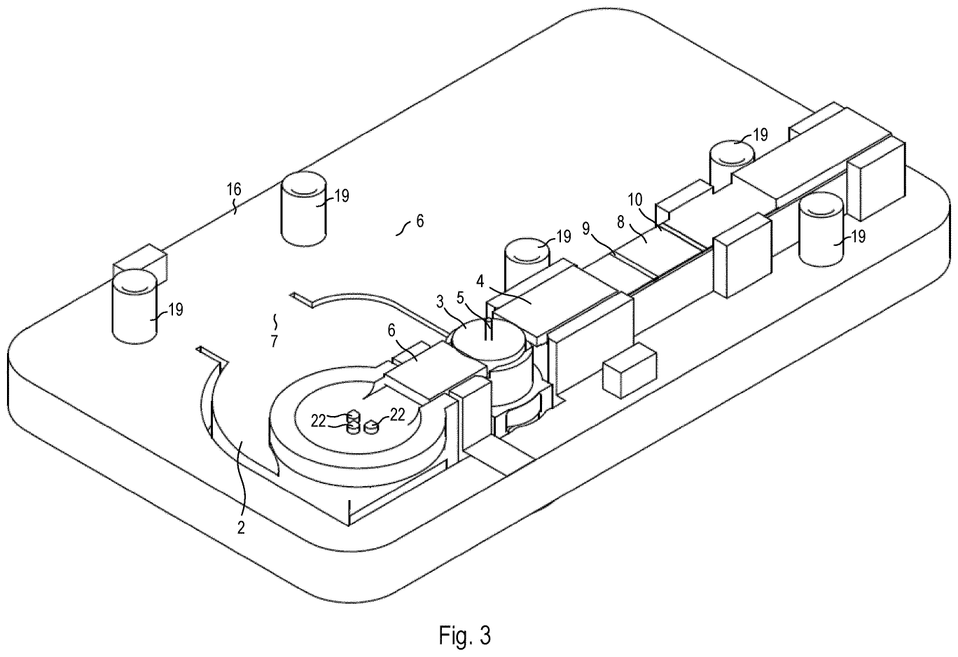

Referring to FIG. 3, a second housing 21 is shown from a third perspective. The second housing 21 is similar to the first housing 1. The view of the second housing 21 comprises three piercing members 22 configured to pierce a seal of the buffer reservoir (not shown) when the actuating member is moved from the first position to the second position. The piercing member 22 may be a needle, a protrusion of the actuating member, or other suitable member.

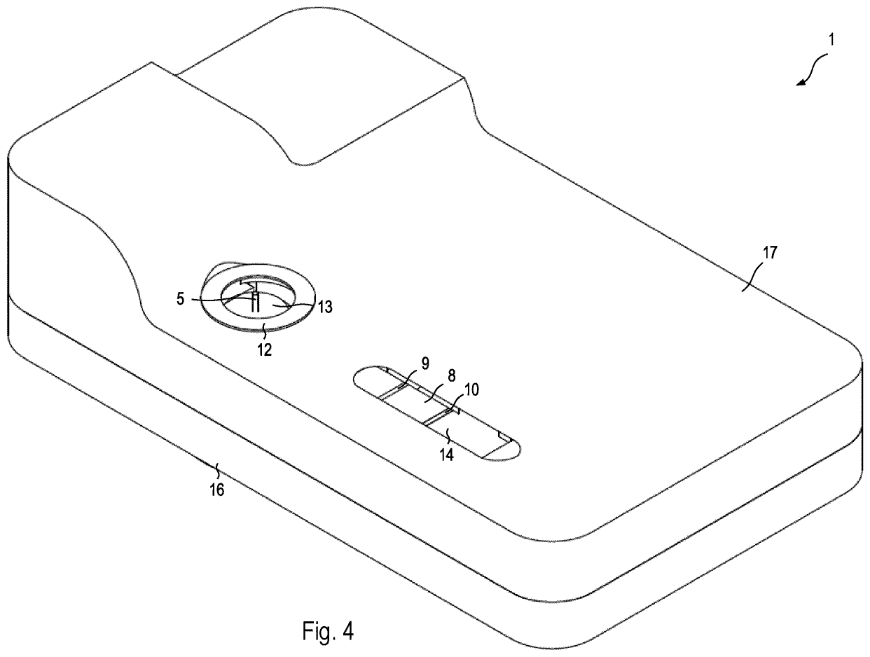

Referring to FIG. 4, an assembled housing 1, 21 is shown by a second housing part 17 connected to a first housing part 16.

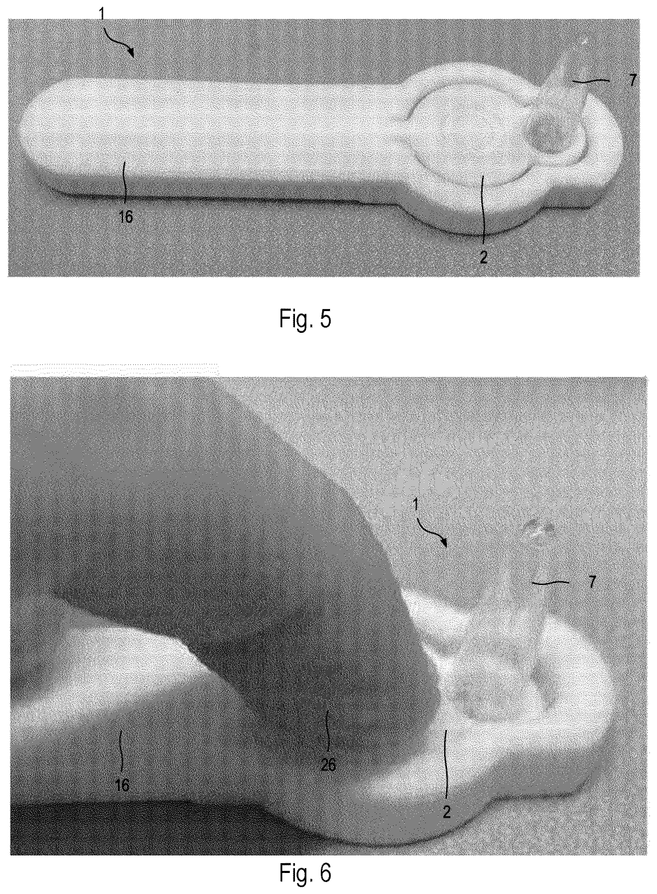

Referring to FIG. 5, a photograph of a third housing 25 is shown. The third housing 25 may be similar to the first housing 1 and second housing 21. An actuating member 2 is shown connected to a buffer reservoir 7. In FIG. 5, the actuating member 2 is in the first position.

Referring to FIG. 6, the actuating member 2 of the third housing 25 may be moved from a first to a second position using a digit 26. In FIG. 6, the actuating member is in the second position.

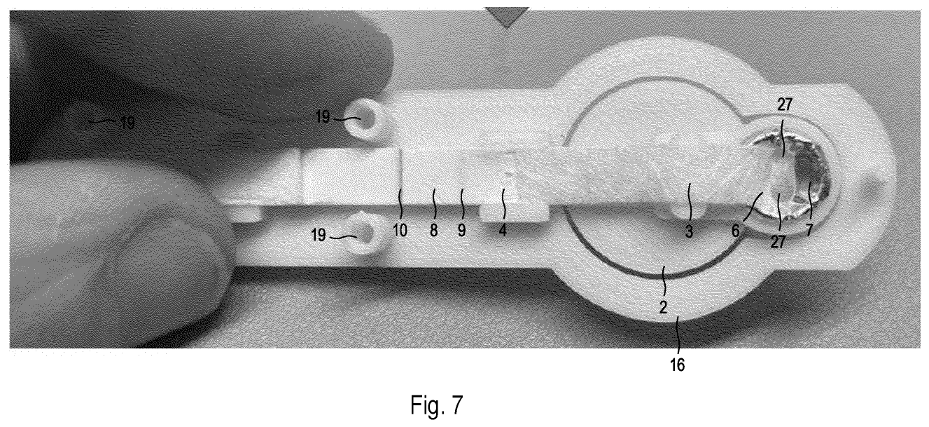

Referring to FIG. 7, a photograph of the inside of the housing 1 is shown after the actuating member has returned to the first position having been moved from the first position to the second position to collect a sample, and to release a buffer on to the buffer component 6 form the buffer reservoir 7. The inside of the housing has a first housing part 16, an actuating member 2, a sample collector 3, a buffer component 6, and a buffer reservoir 7. Puncture holes 27 can be seen in the seal of the buffer reservoir 7.

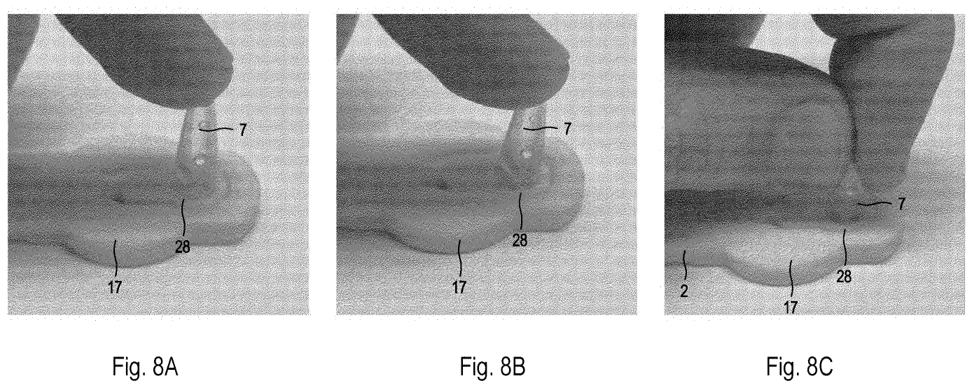

Referring to FIGS. 8A to 8C, the buffer reservoir 7 may be manipulated to allow for the buffer to be transferred from the reservoir 7 to the buffer component 6. There may be a second actuating member 28 which may be attached to the buffer reservoir 7 to allow for the buffer reservoir 7 seal to be broken.

### Needle

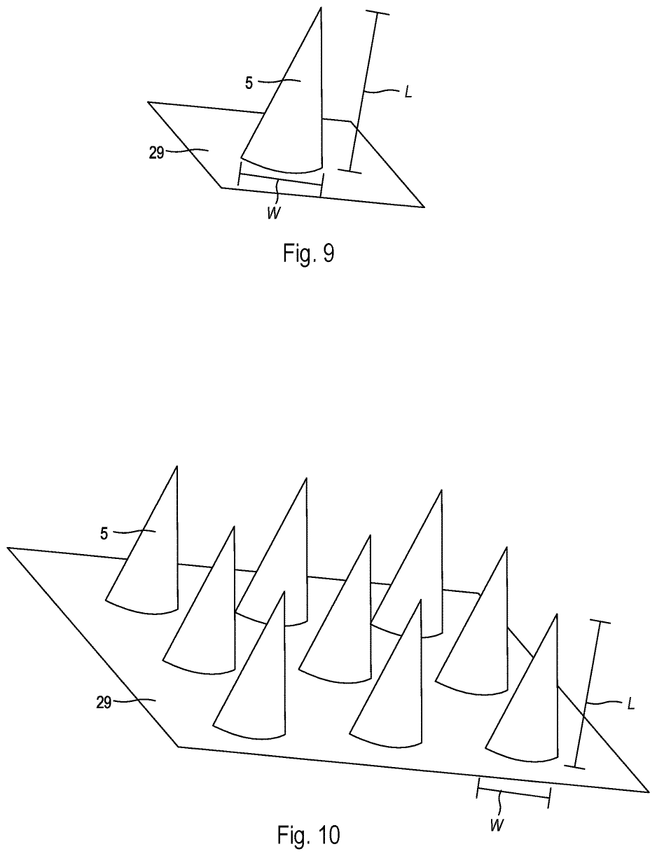

Referring to FIG. 9, the needle 5 may be a micro needle. The microneedle may be solid, and have a length L of between 25 and 2000 μm, and a width W of between 25 and 500 μm at the base. The needle 5 may be any suitable shape. The needle may be a graded needle size, for example, 34 G. The needle 5 may be attached to a needle base 29 which in turn may be attached to the housing, for example, the actuating member 2 of the housing. Referring also to FIG. 10, there may be a plurality of needles 5 attached to the needle bed 29. Referring also to FIG. 11, the needle 5 may be hollow to allow a sample to flow through the centre of the needle 5 onto the sample collector 3.

### Actuator Movement

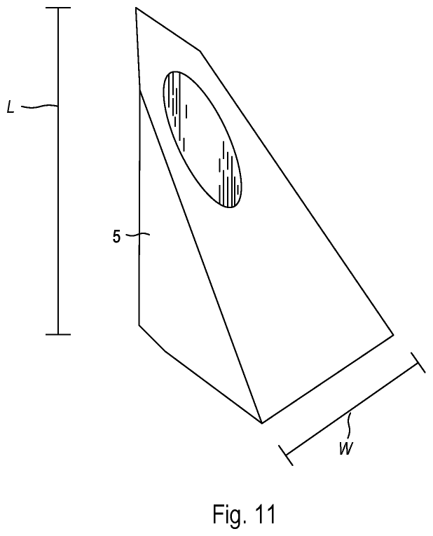

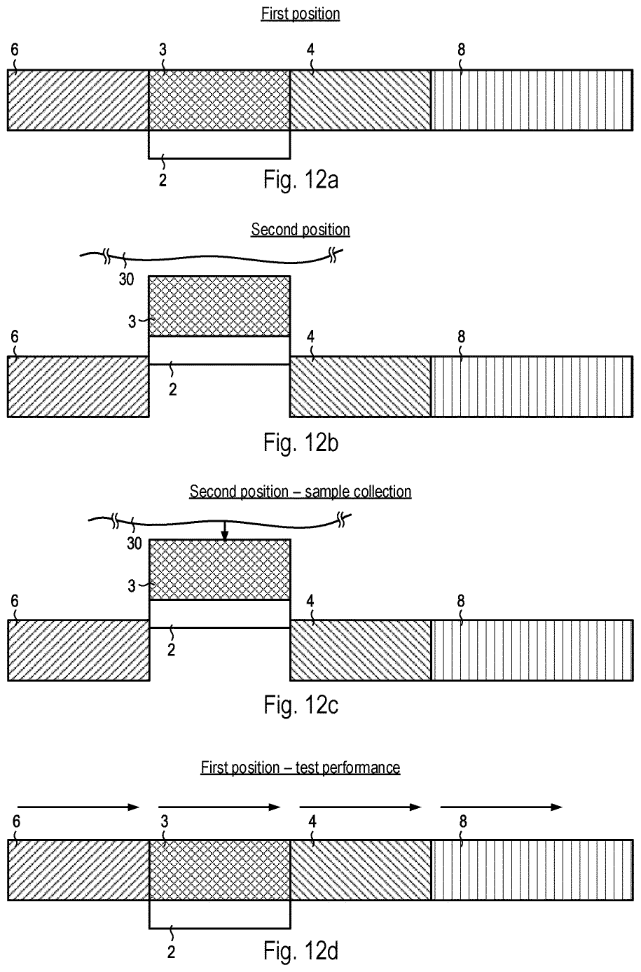

Referring to FIGS. 12a to 12d, the movement of the actuator 2 between the first and the second position is illustrated, and how this movement isolates the sample collector 3 from the assay component 4 while a sample is being collected.

Referring particularly to FIG. 12a, the arrangement of the actuator 2, the sample collector 3, the assay component 4, the buffer component 6 and the test strip 8 when the actuator 2 is in the first position is shown. The arrangement is similar to that of a regular or standard housing for sample collection and testing with the components adjacent to each other, allowing fluid to flow from one component to an adjacent component through, for example, capillary action either directly by the components being in contact with each other, or indirectly via a wick (not shown) or other fluid transportation.

Referring particularly to FIG. 12b, the arrangement of the same components in FIG. 12a when the actuator 2 is in the second position is shown. When moving the actuator 2 from the first position to the second position, the actuator 2 pushes the sample collector 3 closer to a sample source 30 and separates the sample collector 3 from the assay component 4. The separation of the sample collector 3 and the assay component 4 prevents liquid movement from the sample collector 3 to the assay component 4, and optionally, from the buffer component 6 to the sample collector 3. The sample source 30 may be, for example, skin, a tube, or other suitable vessel for temporarily holding a sample for collection.

Referring particularly to FIG. 12c, a sample is transferred from the sample source 30 to the sample collector 3 while the actuator 2 is in the second position. In this way, a sample can be transferred to the sample collector 3 without being transferred on to the assay component 4 or be prematurely diluted by a buffer being transferred from the buffer component 6. Thus, the correct volume or amount of sample can be collected from the sample source 30 into the sample collector 3. As referred to previously, the sample collector 3 may have a structure that defines a predetermined volume of sample to be collected, ensuring that this predetermined volume is not exceeded.

Referring particularly to FIG. 12d, once the sample has been collected into the sample collector 3, the actuator 2 may return to the first position where the test may be performed on the sample. The actuator may return to the first position by a spring (not shown). The actuator 2 may have only one stable state in the first position, that is, the actuator 2 may return to the first position without a new active force being applied such as it being pressed from the second position to return it to the first position. Alternatively, the first position and/or the second position may be a stable state, that is, the actuator 2 may stay in the first or second position without constant force being applied. Once the sample has been collected and the actuator 2 returned to the first position, buffer fluid may flow from the buffer component 6 to the sample collector 3, and then both the buffer fluid and the sample may flow to the test component where a test may be performed. If the test is a lateral flow test, there may be a test strip 8 adjacent to the assay component for performing the test. Alternatively, the assay component may be used for the appropriate test, for example, ELISA.

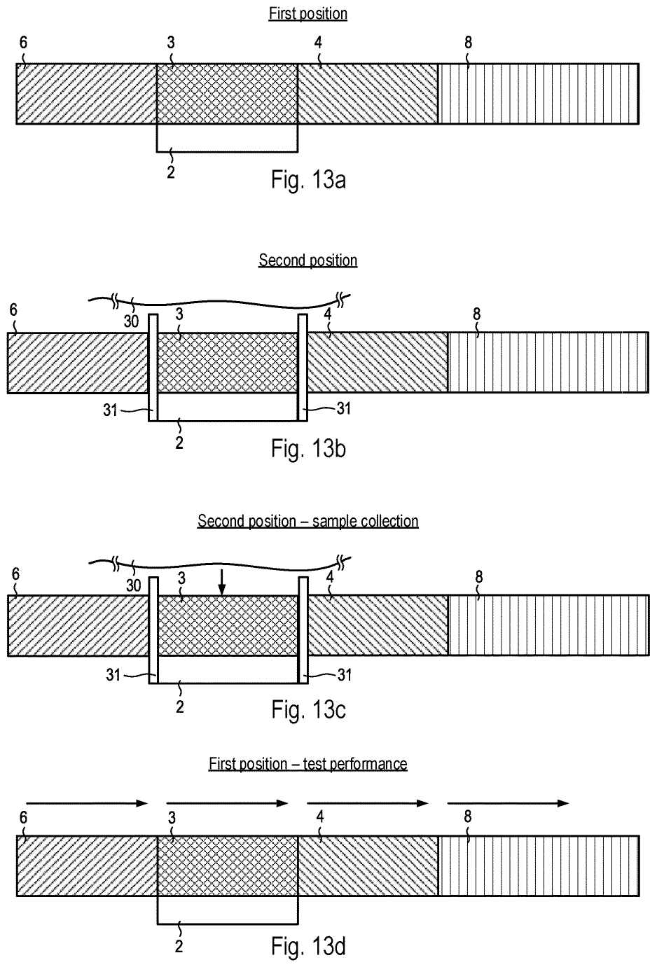

Referring to FIGS. 13a to 13d, the components of the housing 1 for sample collection are the similar to those in FIGS. 12a to 12d. Referring particularly to FIG. 13b, in the second position, the separation of the sample collector 3, and the assay component 4, and optionally the buffer component 6, is performed by a barrier 31 preventing fluid or liquid from passing between the components and collector. There may be more than one barrier 31. The barrier 31 may be formed from one or multiple parts. Referring particularly to FIG. 13d, the barrier 31 is automatically removed from between the sample collector 3 and the assay component 4, and optionally the buffer component 6, when the actuator 2 is returned to the first position. The barrier 31 removal may allow for the test to be performed.

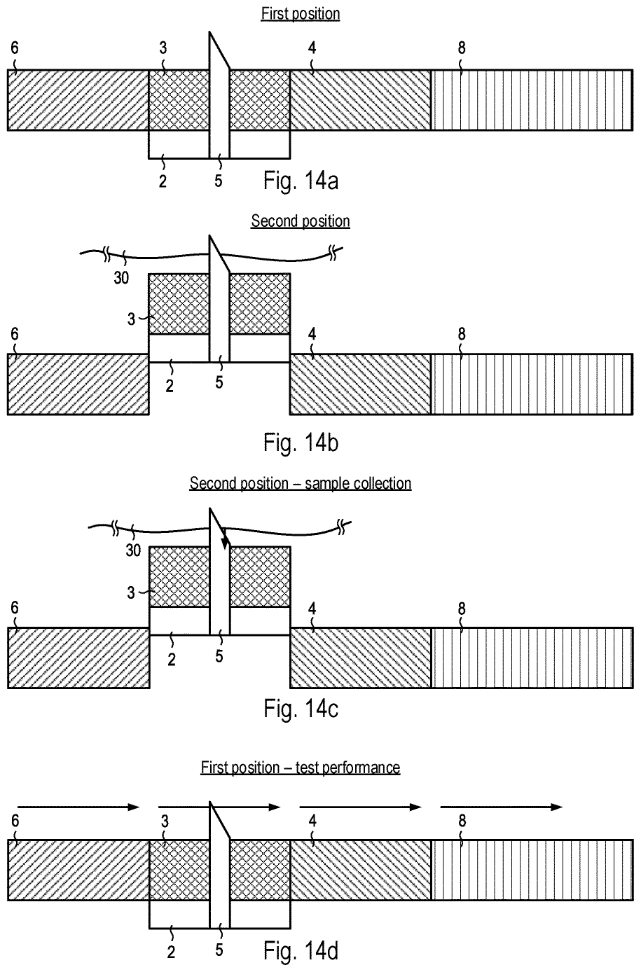

Referring to FIGS. 14a to 14d, as with FIGS. 13a to 13d, the components of the housing 1 for sample collection are the similar to those in FIGS. 12a to 12d. Referring particularly to FIG. 14b, a needle 5 may be arranged adjacent to, or arranged to pierce the sample collector 3. The needle 5 may be attached to the actuator 2. The needle 5 may move together with the actuator 2 and the sample collector 3 into the second position. In the first position the needle 5 is disposed within the housing 1 and in the second position the needle 5 protrudes from an aperture (not shown) in the housing (not shown). Referring particularly to FIG. 14c, with the needle 5 protruding from the housing, the needle may puncture the sample source 30 (e.g. the skin of a patient), and allow the sample (e.g. blood) to flow from the sample source 30 to the sample collector 3. Referring particularly to FIG. 14d, when the actuator 2 is returned to the first position, the test may be performed.

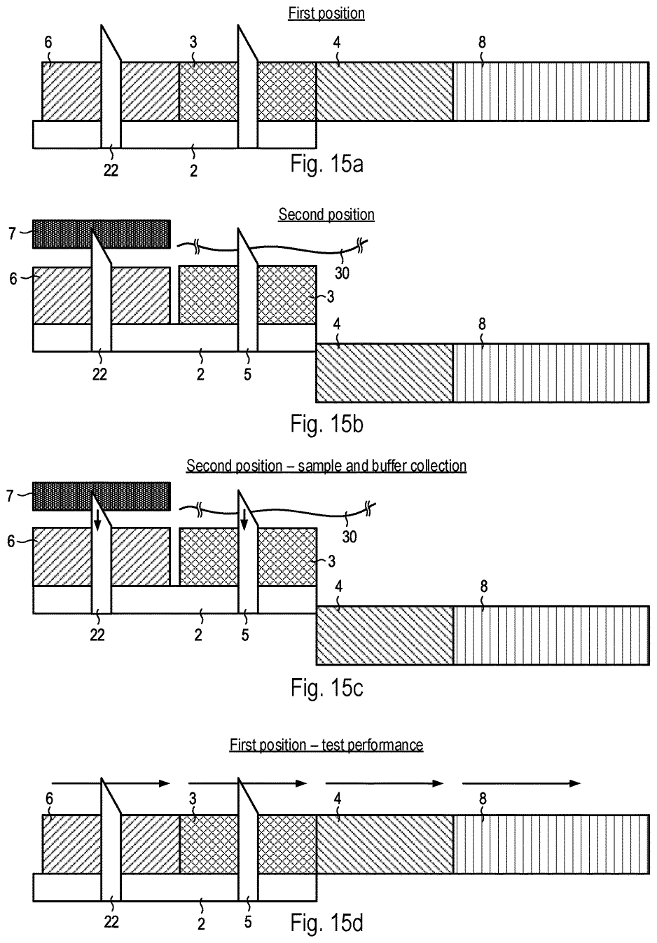

Referring to FIGS. 15a to 15d, the housing may further include a piercing member 22. The piercing member 22 may be attached to the actuating member 2 and/or be configured to move relative to the actuating member 2. The piercing member 22 may be adjacent to the buffer component 6. Referring particularly to FIGS. 15b and 15c, when the actuating member 2 is moved to the second position, the piercing member 22 is configured to break a seal (not shown) in the buffer reservoir 7, allowing buffer to flow from the reservoir 7 to the buffer component 6. This may happen at the same time as the needle 5 pierces the sample source 30 to collect the sample in the sample collector 3. Thus, with one action, the buffer may be transferred to the buffer component 6 and the sample may be transferred to the sample collector 3. In the second position, the buffer component 6 and the sample collector 3 are separated by a space of a barrier 31 (see FIGS. 13a to 13d) to prevent the flow of the buffer into the sample collector 3. Alternatively, the piercing member 22 may be arranged on the second housing part 17 and a seal on the buffer reservoir 7 is pierced by the reservoir 7 being moved with the actuating member 2 to engage with the piercing member 22 on the second housing part 17. Referring particularly to FIG. 15d, when the actuator 2 is returned to the first position, the test may be performed. The buffer reservoir 7 and the sample source 30 may be pierced at the same time.

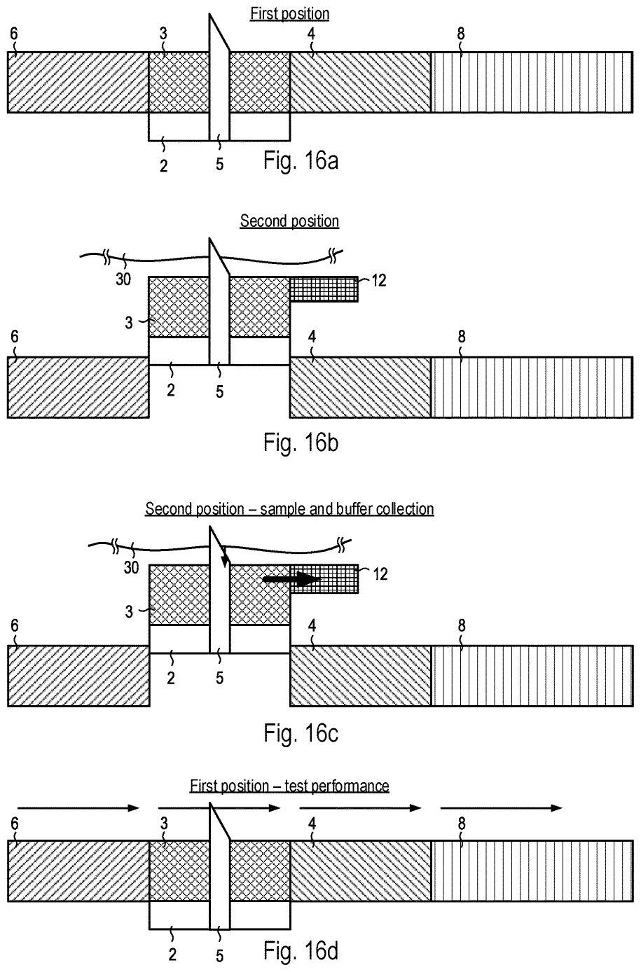

Referring to FIGS. 16a to 16d, an arrangement of components similar to those in FIGS. 14a to 14d is shown. Referring particularly to FIGS. 16b and 16c, in the second position, the actuator 2 moves the sample collector 3 to a position configured to collect a sample, and also to be in fluid (e.g. liquid) communication with an excess sample collector 12 configured to take excess sample from the sample collector 3 once the sample collector 3, for example, when the sample collector 3 has been filled with a predetermined volume of sample. This may ensure that a predetermined volume of sample is used for the test, with any excess being transferred from the sample collector 3 to the excess sample collector 12. Referring particularly to FIG. 16d, the excess sample collector 12 is isolated from the sample collector 12 in the first position, this ensures that the excess sample is not transferred from the excess sample collector 12 to the sample collector 3. When the actuator 2 is returned to the first position, the test may be performed.

The housing 1 may also include a blood separation filter (not shown), for example, a blood separation membrane FR-1 (0.35) from www.mdimembrane.com which is capable of separating red blood cells from serum in blood. The blood separation filter (not shown) may be disposed between the sample source 30 and the assay component 4, for example between the assay component 4 and the sample collector 3, or between the sample source 30 and the sample collector 3. With such an arrangement, an assay may be performed on blood serum rather than blood.

The components in the housing 1 described above may be used in any suitable arrangement, for example, the excess sample collector 12 may be used in a housing where there is no needle 5, because a needle 5 may not be required for all sample collections. Likewise, the barriers 31, or other suitable isolation method or arrangement, may be used in any of the described arrangements. The arrangements of components inside a housing 1 described above ensure that the correct volume of a sample is collected, and that the test is performed in the correct order, so that buffer, samples and/or reagents are not wasted, with one motion of an actuator, for example it being pressed by a digit to move it to the second position and released, returning it to the first position. Thus, the housing can be used to perform accurate tests by inexperienced practitioners or members of the public.

### Quantitative Assay

The housing 1 described above may be used to perform sample collection and a subsequent test. One test which may be required to be performed regularly by members of the public is a test for the absence of vitamin K in humans, particularly neonates. Thus, there is a need for a reliable test for the absence of vitamin K in a home environment, outside of a hospital or clinic.

Lateral flow tests are ideal tests to be performed at home as they are simple to use. Lateral flow tests are predominantly used to indicate the presence or absence of a particular molecule in a binary result. However, as will now be explained, for a prothrombin induced by vitamin K absence-II (PIVKA-II) assay performed using a lateral flow test kit, the intensity of a test indicator (for example, in absorbance (Au)) can be used to indicate a quantity of an analyte, for example by indicating a range of value or concentrations of an analyte.

A suitable off the shelf lateral flow development kit (for example: https://www.abcam.com/universal-lateral-flow-assay-kit-ab270537.html) and an off-the-shelf PIVKA-II 3C10 antibody may be used to identify PIVKA-II from patient serum samples using 1 μl of sample, at the required concentration for a useful clinical test.

The test indicator 10 (test line) of the lateral flow assay test strip 8 may be read using a conventional phone camera and the image processed using standard image processing software to calculate intensity, normalised to the background, or to the control indicator 9.

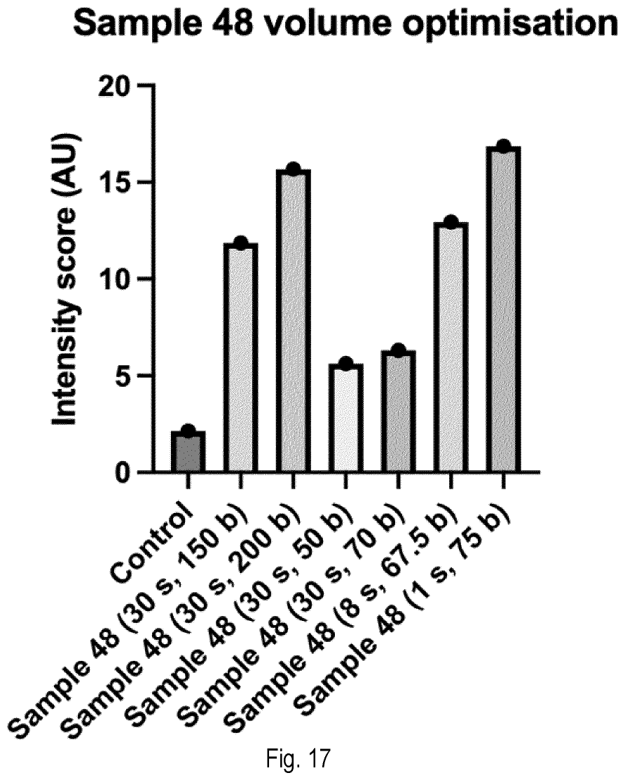

Referring to FIG. 17, a bar chart shows the intensity score in absorbance (Au) for a control test and six samples. Each of the six samples are the same clinical sample, at different volumes and concentrations of the sample. For example, the sample on the left hand side (“Sample 48 (30 s, 150 b))” denotes 30 μm of the sample, and 150 μm of the buffer.

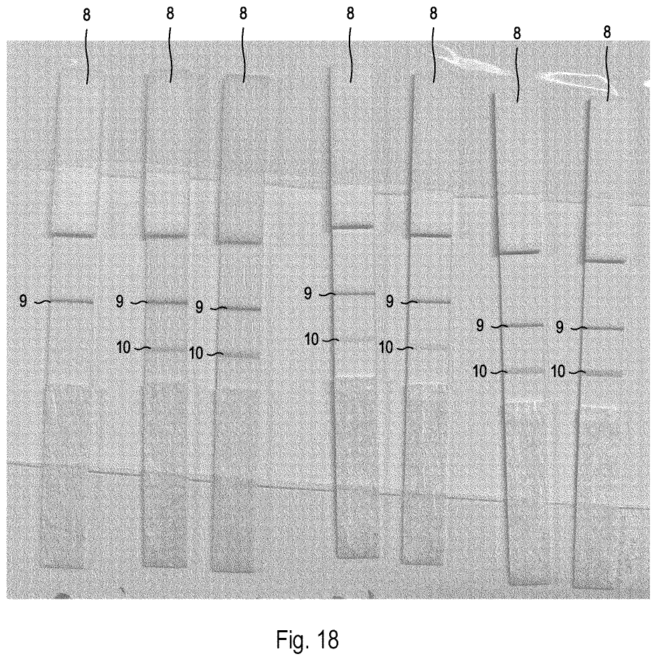

Referring to FIG. 18, a photograph of seven lateral flow test strips 8 are shown each having control indicators 9 and varying strengths of test indicators 10. The left hand side test strip 8 is a control strip with no PIVKA-II present in the sample, the remaining test strips show the results for Sample 48 (30 s, 150 b), Sample 48 (30 s, 200 b), Sample 48 (30 s, 50 b), Sample 48 (30 s, 70 b), Sample 48 (8 s, 67.5 b), and Sample 48 (1 s, 75 b), the absorbance results of which are shown in FIG. 17. Thus, it is possible to identify PIVKA-II using a 1 μL sample using a lateral flow assay.

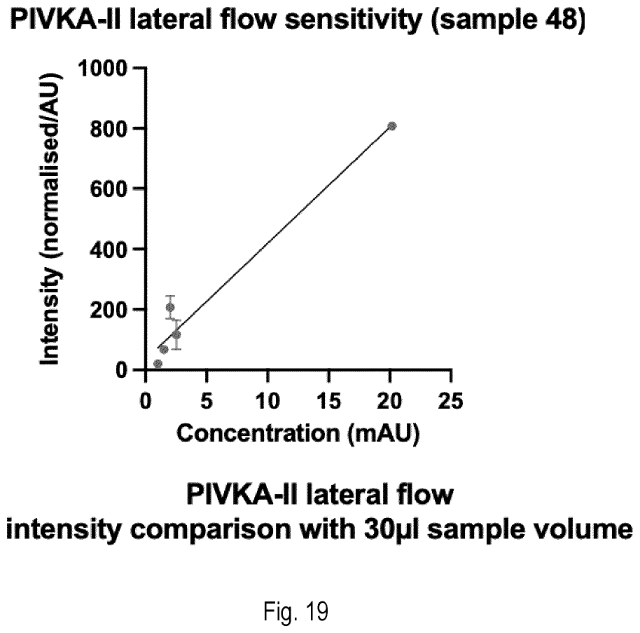

Referring to FIG. 19, a scatter-chart of normalised intensity (normalised/Au) of the test indicator 10 with respect to the control indicator 9 of a PIVKA-II lateral flow assay is shown for concentrations (mAU) between near zero and 20 milli-absorbance (mAU) shows that the intensity of the test indicator 10 increased with increasing concentration.

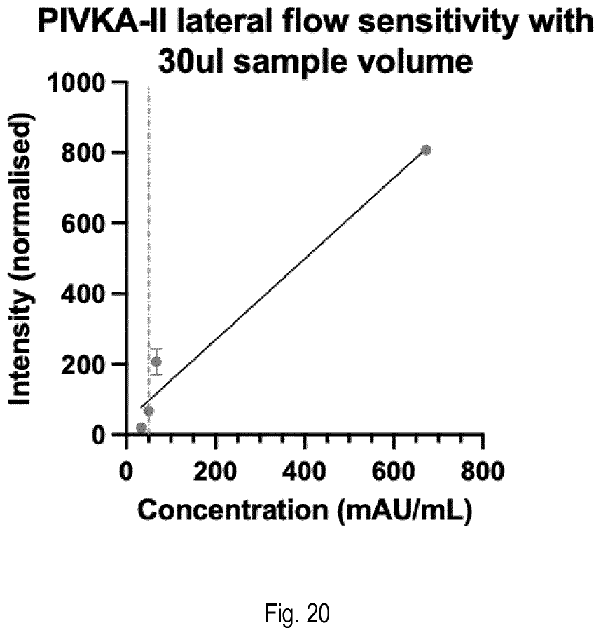

Referring to FIG. 20, a scatter-chart of normalised colour intensity of the test indicator 10 with respect to the control indicator 9 of a PIVKA-II lateral flow assay is shown for concentrations of milli-absorbance per millilitre (mAU/mL) between near zero and around 700 mAU/mL for a 30 μl sample. The dashed line at 51 mAU/mL indicates the maximum normal clinical range for PIVKA-II, that is, above this value indicates a vitamin K deficiency.

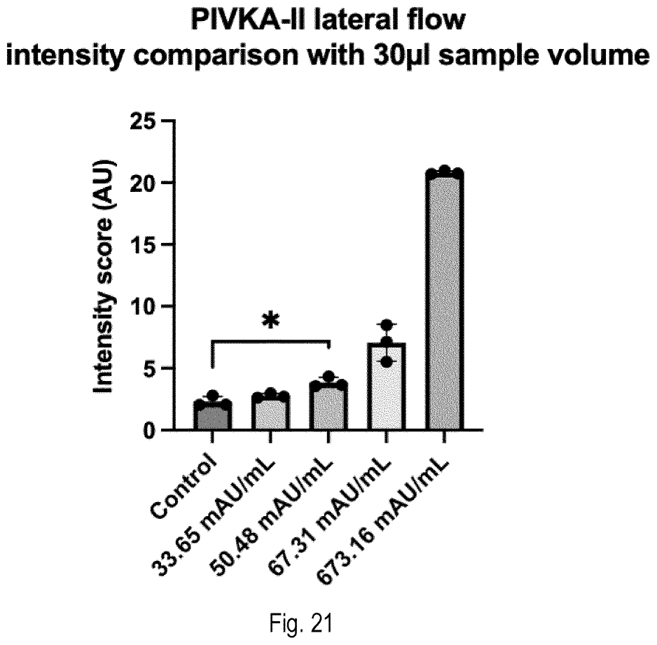

Referring to FIG. 21, a bar chart showing the normalised intensity score (the intensity of the test indicator 10 with respect to the control indicator 9) in absorbance (AU) for a control lateral flow test where the control contains no PIVKA-II, and four samples containing difference concentrations of PIVKA-II (33.65 mAU/mL, 50.48 mAU/mL, 67.31 mAU/mL, 673.16 mAU/mL). The chart shows a statistically significant difference in normalised intensity between the control, 33.65 mAU/mL and 50.48 mAU/mL verses both the 67.31 mAU/mL, 673.16 mAU/mL concentrations. It is therefore possible to use the normalised intensity of the lateral flow test indicator 10, it is possible to identify at least a range concentration of PIVKA-II using a lateral flow test. Thus, the results presented in these Figures show that with a 1 μl sample volume it is possible to identify 250 mAU/mL (ng/ml). When using a 30 μl volume, it is possible to detect a 51 ng/ml concentration-the maximum normal clinical range. It is possible to detect a 51 ng/mL concentration with much lower sample volumes, for example, 20 μl, 10 μl, 5 μl, and 1 μl.

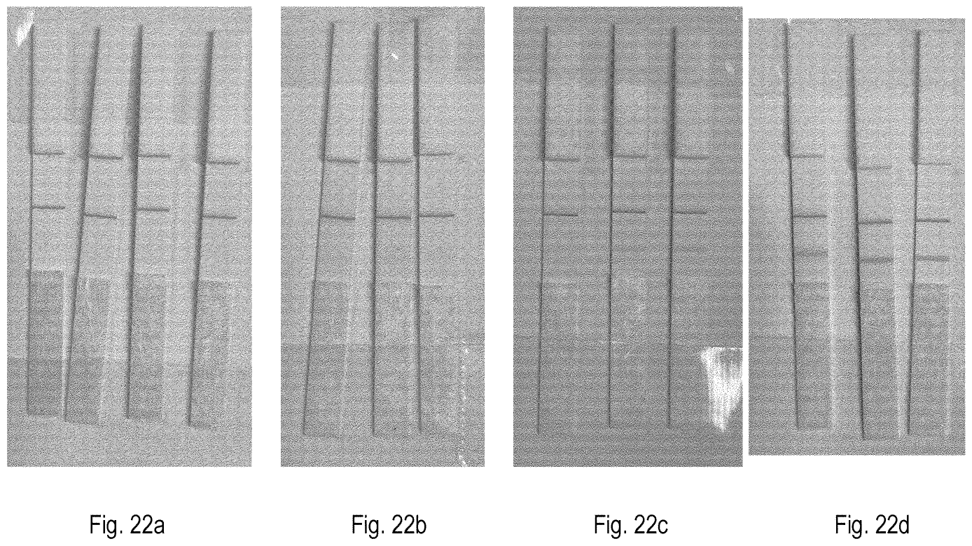

Referring to FIG. 22a, a photograph of four control lateral flow test for PIVKA-II show clear control indicators 9 and faint test indicators 10. Referring to FIG. 22b, a photograph of three lateral flow test strips 8 for PIVKA-II at a concentration of 125 mAU/mL show clear control indicators 9 and faint test indicators 10. Referring to FIG. 22c, a photograph of three lateral flow test strips 8 for PIVKA-II at a concentration of 250 mAU/mL show clear control indicators 9 and test indicators 10 which are clearer than those in FIG. 22b. Referring to FIG. 22d, a photograph of three lateral flow test strips 8 for PIVKA-II at a concentration of 2500 mAU/mL show clear control indicators 9 and clear test indicators.

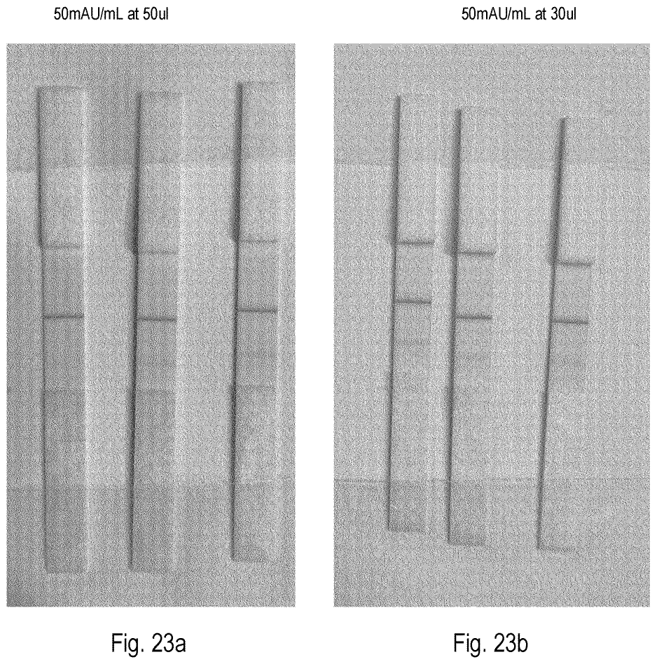

Referring to FIG. 23a, a photograph of three lateral flow test strips 8 having been performed with a 50 μl sample at a concentration of 50 mAU/mL. Referring to FIG. 23b a photograph of three lateral flow test strips 8 having been performed with a 30 μl sample at a concentration of 50 mAU/mL. Each of FIGS. 23a and 23b have clear control indicators 9, and faint control indicators 10, demonstrating that the test is sensitive to the upper normal range of PIVKA-II. That is, there is not an indication of a presence of PIVKA-II when below the maximum normal clinical range.

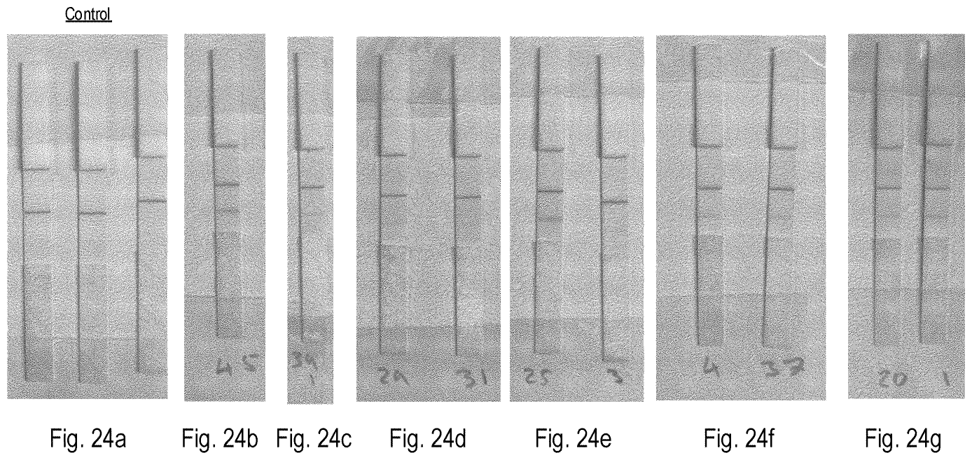

Referring to FIG. 24a, a photograph of three control lateral flow test for PIVKA-II show clear control indicators 9 and faint test indicators 10. Referring to FIGS. 24b to 24g, photographs of lateral flow test strips having has tests completed for a range of different samples are shown. The samples have different concentrations of vitamin K, which can clearly be seen in the test results. For example, FIG. 24e left hand side test strip, labelled 25, shows a test for a high concentration of PIVKA-II (therefore low vitamin K), whereas FIG. 24 d right hand side test strip, labelled 31, shows a low concentration of PIVKA-II.

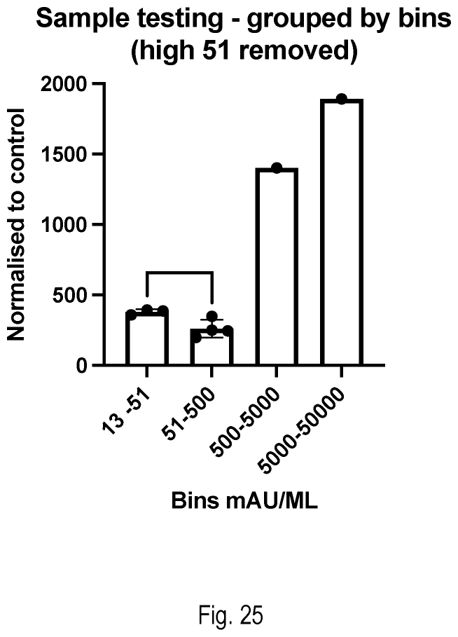

Referring to FIG. 25, a bar chart of intensity (arbitrary units) normalised to control for four different concentrations of PIVKA-II-between 13 and 51 mAU/mL, between 51 and 500 mAU/mL, between 500 and 5,000 mAU/mL, and between 5,000 and 50,000 mAU/mL. There is a significant difference between the 13 to 51 mAU/mL and 51 to 500 mAU/mL concentrations.

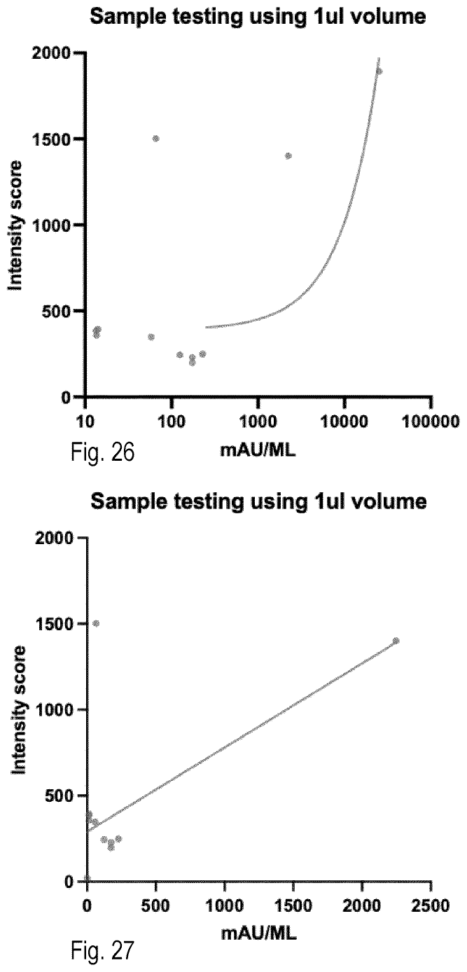

Referring to FIGS. 26 and 27, a scatter chart of intensity score (arbitrary units) against a range of concentrations shows that intensity scores increase with increased concentrations of PIVKA-II for 1 μL volumes.

### Modifications

It will be appreciated that various modifications may be made to the embodiments hereinbefore described. Such modifications may involve equivalent and other features which are already known in the design, manufacture and use of housings for sample collection, quantitative lateral flow assays and component parts thereof and which may be used instead of or in addition to features already described herein. Features of one embodiment may be replaced or supplemented by features of another embodiment.

Although claims have been formulated in this application to particular combinations of features, it should be understood that the scope of the disclosure of the present invention also includes any novel features or any novel combination of features disclosed herein either explicitly or implicitly or any generalization thereof, whether or not it relates to the same invention as presently claimed in any claim and whether or not it mitigates any or all of the same technical problems as does the present invention. The applicants hereby give notice that new claims may be formulated to such features and/or combinations of such features during the prosecution of the present application or of any further application derived therefrom.

1. A housing for sample collection, the housing comprising:
an actuating member configured to move between a first position and a second position;
a sample collector; and
an assay component;
wherein in the first position the sample collector is in contact with the assay component and in the second position the sample collector is separated from the assay component and configured to collect a sample.

2. The housing of claim 1, further comprising a needle, wherein in the first position the needle is disposed within the housing and in the second position the needle protrudes from an aperture in the housing.

3. The housing of claim 1 wherein the sample collector is a pad.

4. The housing of claim 1 further comprising:
an excess sample collector, wherein the sample collector is configured to hold a predetermined volume of liquid and when the actuator is in the second position, the excess sample collector is configured to collect or absorb excess liquid from the sample collector once the sample collector holds its predetermined volume, and wherein the sample collector and the excess sample collector are separated from the assay component, and in the first position, the sample collector is separated from the excess sample collector.

5. A housing for sample collection, the housing comprising:
an actuating member configured to move between a first position and a second position;
a piercing member; and
a needle;
wherein in the first position the needle is disposed within the housing and in the second position the needle protrudes from an aperture in the housing, and wherein the piercing member is configured to pierce a seal of a fluid reservoir upon movement from the first position to the second position.

6. The housing of claim 2 wherein the needle is disposed on a surface of the actuating member.

7. The housing of claim 5 further comprising a sample collector configured to receive bodily fluid.

8. The housing of claim 7 wherein moving the actuating member from the first position to the second position causes the needle to pierce the sample collector, or causes the needle to move past the sample collector.

9. The housing of claim 1 wherein the housing comprises a base and a cover, and wherein the actuating member is attached to, and moveable relative to, the base.

10. The housing of claim 1 when dependent on claim 5 further comprising an adhesive arranged on a surface of the housing.

11. The housing of claim 9 further comprising an excess sample collector configured to receive bodily fluid from the sample collector or from a body.

12. The housing of claim 1 further comprising a sample collector aperture or a sample collector window in a surface of the housing configured to allow visual inspection of the sample collector.

13. The housing of claim 1 further comprising a buffer fluid reservoir comprising a seal.

14. The housing of claim 1 further comprising a buffer component configured to receive fluid from a, or the, buffer fluid reservoir.

15. The housing of claim 2 when dependent on any of claim 5 wherein once the actuating member has reached the second position, an end-stop or a spring causes the needle to move to a needle position between the needle position in the first position and the needle position in the second position.

16. The housing of claim 13, wherein when the housing comprises a sample collector configured to receive a bodily fluid and at least one of an assay component or a buffer component configured to receive fluid from the buffer fluid reservoir, in the first position, the reservoir is in contact with the assay component or the pad and in the second position, there is a space between the reservoir and the assay component or the buffer component.

17. The housing of claim 1 further comprising a lateral flow test strip viewing aperture or window configured to allow the viewing of a lateral flow test strip.

18. A test device comprising an assay component disposed in a housing according to claim 5.

19. The test device of claim 18 wherein the assay component is configured to detect the presence or absence of a vitamin.

20. (canceled)

21. A method of determining a concentration range of vitamin K in a sample using a prothrombin induced by vitamin K absence-II (PIVKA-II) lateral flow assay, the method comprising:
performing a PIVKA-II lateral flow test on the sample;
measuring the intensity of the test line on the lateral flow test strip; and determining a concentration range of vitamin K of the sample based on a test result indicator.

22. (canceled)

23. (canceled)

24. (canceled)

25. (canceled)
